(19) United States
(12) Patent Application Publication
(10) Pub. No.: US 2002/0040047 A1
(43) Pub. Date: Apr. 4, 2002

(54) Novel Use

(76) Inventors: Edward Raymond Halstead Walker, Cheshire (GB); Roger Metcalf, Cheshire (GB); Stephen John Foster, Cheshire (GB)

Correspondence Address: PILLSBURY WINTHROP LLP, 1600 TYSONS BOULEVARD, MCLEAN, VA, 22102, US

(21) Appl. No.: 09/729,983

(22) Filed: Dec. 6, 2000

(30) Foreign Application Priority Data
Aug. 2, 2000 (GB) 0018829.2

(51) Int. Cl. A61K 31/55; A61K 31/4709; A61K 31/404

(52) U.S. Cl. 514/418; 514/312; 514/212.07

(57) ABSTRACT

The invention relates to the use of 4-aryl-4-hydroxy-tetrahydropyrans and 3-aryl-3-hydroxy-tetrahydrofurans for the treatment or prevention of chronic obstructive pulmonary disease.

The present invention relates to the use of 4-aryl-4-hydroxy-tetrahydropyrans and 3-aryl-3-hydroxy-tetrahydrofurans for the treatment or prevention of chronic obstructive pulmonary disease (hereafter referred to as COPD).

COPD is a prolonged or persistent condition of respiratory dysfunction resulting in oxygenation or carbon dioxide elimination at a rate that is not sufficient to meet the demands of the body. It may be severe enough to impair or threaten the function of vital organs. It is the fourth leading cause of death in the United States, affecting approximately 10% of all adults.

U.S. Pat. No. 5,512,594 discloses certain ether derivatives having 5-LO inhibitor activity.

In a first aspect the invention provides the use of an ether derivative of a compound of formula (I):

Q¹—X—Ar—Q² (I)

wherein

Q¹ is a 9-, 10- or 11-membered bicyclic heterocyclic moiety containing one or two nitrogen heteroatoms and optionally containing a further heteroatom selected from nitrogen, oxygen and sulphur, and Q¹ may optionally bear up to four substituents selected from halogeno, hydroxy, cyano, formyl, oxo, thioxo, (1-4C) alkyl, (3-4C) alkenyl, (3-4C) alkynyl, (1-4C) alkoxy, fluoro-(1-4C) alkyl, hydroxy-(1-4C) alkyl, (2-5C) alkanoyl, phenyl, benzoyl and benzyl, and wherein said phenyl, benzoyl and benzyl substituents may optionally bear one or two substituents selected from halogeno, (1-4C) alkyl and (1 -4C) alkoxy;

X is oxy, thio, sulphinyl or sulphonyl;

Ar is phenylene, pyridinediyl, pyrimidinediyl, thiophenediyl, furandiyl, thiazolediyl, oxazolediyl, thiadiazolediyl or oxadiazolediyl which may optionally bear one or two substituents selected from halogeno, cyano, trifluoromethyl, hydroxy, amino, (1-4C) alkyl, (1-4C) alkoxy, (1-4C) alkylamino and di-(1-4C) alkylamino;

and Q² is selected from the groups of the formulae II and III:

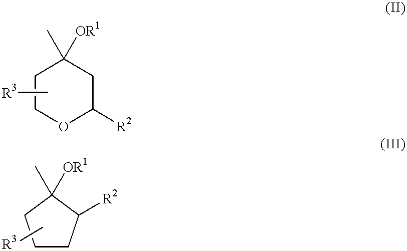

wherein

R¹ is hydrogen, (2-5C) alkanoyl or benzoyl, and wherein said benzoyl group may optionally bear one or two substituents selected from halogeno, (1-4C) alkyl and (1-4C) alkoxy;

R² is (1-4C) alkyl; and

R³ is hydrogen or (1-4C) alkyl;

or R² and R³ are linked to form a methylene, vinylene, ethylene or trimethylene group;

or a metabolite thereof, or a pharmaceutically acceptable salt thereof, for the manufacture of a medicament for use in the treatment or prevention of COPD in a mammal (especially a human).

In this specification the generic term “alkyl” includes both straight-chain and branched-chain alkyl groups. However references to individual alkyl groups such as “propyl” are specific for the straight-chain version only and references to individual branched-chain alkyl groups such as “isopropyl” are specific for the branched-chain version only. An analogous convention applies to other generic terms.

It is to be understood that, insofar as certain of the compounds of the formula (I) defined above may exhibit the phenomenon of tautomerism and any formula drawing presented herein may represent only one of the possible tautomeric forms, the invention includes in its definition any tautomeric form of a compound of the formula (I) which possesses the property of treating or preventing COPD and is not to be limited merely to any one tautomeric form utilised within the formulae drawings.

It is further to be understood that, insofar as certain of the compounds of formula (I) defined may exist in optically active or racemic forms by virtue of one or more asymmetric carbon atoms, the invention includes in its definition any such optically active or racemic form which possesses the property of treating or preventing COPD. The synthesis of optically active forms may be carried out by standard techniques of organic chemistry well known in the art, for example by synthesis from optically active starting materials or by resolution of a racemic form.

Suitable values for the generic terms referred to above include those set out below.

A suitable value for Q¹ when it is a 9-membered bicyclic heterocyclic moiety containing one or two nitrogen heteroatoms and optionally containing a fisher heteroatom selected from nitrogen, oxygen and sulphur is, for example, a benzo-fused heterocyclic moiety or a hydrogenated derivative thereof such as indolyl, indolinyl, isoindolyl, isoindolinyl, indolizinyl, benzimidazolyl, 2,3-dihydrobeazimidazolyl, 1H-indazolyl, 2,3-dihydro-1H-indazolyl, benzoxazolyl, 2,3-dihydrobenzoxazolyl, benzo[c]isoxazolyl, benzo[d]isoxazolyl, 2,3-dihydrobenzo[d]isoxazolyl, benzothiazolyl, 2,3-dihydrobenzothiazolyl, benzo[c]isothiazolyl, benzo[d]isothiazolyl and 2,3-dihydrobenzo[d]isothiazolyl, or, for example, a pyrido-fused heterocyclic moiety or a hydrogenated derivative thereof such as 1H-pyrrolo[2,3-b]pyridyl, 2,3-hydro-1H-pyrrolo[2,3-b]pyridyl, 1H-pyrrolo[2,3-c]pyridyl, 2,3-dihydro-1H-pyrrolo[2,3-c]pyridyl, 1H-imidazo[4,5-b]pyridyl, 2,3-dihydro-1H-imidazo[4,5-b]pyridyl, 1H-imidazo[4,5-c]pyridyl and 2,3-dihydro-1H-imidazo[4,5-c]pyridyl.

A suitable value for Q¹ when it is a 10-membered bicyclic heterocyclic moiety containing one or two nitrogen heteroatoms and optionally containing a further heteroatom selected from nitrogen, oxygen and sulphur is, for example, a 10-membered benzo-fused heterocyclic moiety such as quinolyl, isoquinolyl, cinnolinyl, quinazolinyl, quinoxalinyl, 4H-1,4-benzoxazinyl or 4H-1,4-benzothiazinyl, or a hydrogenated derivative thereof such as 1,2-dihydroquinolyl, 1,2,3,4-tetrahydroquinolyl, 1,2-dihydroisoquinolyl, 1,2,3,4-tetrahydroquinazolinyl, 2,3-dihydro4H-1,4-benzoxazinyl or 2,3-dihydro-4H-1,4-benzothiazinyl; or, for example, a 10-membered pyrido-fused heterocyclic moiety such as 1,7-naphthyridinyl, 1,8-naphthyridinyl, pyrido[2,3-d]pyrinidinyl, pyrido[2,3-b]pyrazinyl, 4H-pyrido[3,2-b][1,4]oxazinyl and 4H-pyrido[3,2-b][1,4]thiazinyl, or a hydrogenated derivative thereof.

A suitable value for Q¹ when it is an 11-membered bicyclic heterocyclic moiety containing one or two nitrogen heteroatoms and optionally containing a further heteroatom selected from nitrogen, oxygen and sulphur is, for example, an 11-membered benzo-fused heterocyclic moiety such as 1H-benzo[b]azepine, or a hydrogenated derivative thereof such as 2,3,4,5-tetrahydro-1H-benzo[b]azepine.

The heterocyclic moiety may be attached through any available position including from either of the two rings of the bicyclic heterocyclic moiety and including through an available nitrogen atom. The heterocyclic moiety may bear a suitable substituent such as, for example, a (1-4C) alkyl, (3-4C) alkenyl, (3-4C) alkynyl, fluoro-(1-4C) alkyl, phenyl, benzoyl or benzyl substituent on an available nitrogen atom.

Suitable values for substituents which may be present on Q¹ or Ar, on the phenyl substituent on Q¹, on any of the substituents on Q¹ which contain a phenyl group, or on R¹ when is benzoyl include, for example:

| | |
|---|---|
| for halogeno: | fluoro, chloro, bromo and iodo; |
| for (1-4C) alkyl: | methyl, ethyl, propyl, isopropyl, butyl, |
| | isobutyl, sec-butyl and tert-butyl; |
| for (3-4C) alkenyl: | allyl, 2-butenyl and 3-butenyl; |
| for (3-4C) alkynyl: | 2-propynyl and 2-butynyl; |
| for (1-4C) alkoxy: | methoxy, ethoxy, propoxy, |
| | isopropoxy and butoxy; |
| for fluoro-(1-4C) alkyl: | fluoromethyl, difluoromethyl, |
| | trifluoromethyl, 2-fluoroethyl, 2,2,2- |
| | trifluoroethyl and pentafluoroethyl; |
| for hydroxy-(1-4C) alkyl: | hydroxymethyl, 2-hydroxyethyl and |
| | 3-hydroxypropyl; |
| for (2-5C) alkanoyl: | acetyl, propionyl and butyryl; |
| for (1-4C) alkylamino: | methylamino, ethylamino and |
| | propylamino; and |
| for di-(1-4C) alkylamino: | dimethylamino, diethylamino and N- |
| | ethyl-N-methylamino. |

A suitable value for Ar when it is phenylene is, for example, 1,3- or 1,4-phenylene.

A suitable value for Ar when it is pyridinediyl, pyrimidinediyl, thiophenediyl, furandiyl, thiazolediyl, oxazolediyl, thiadiazolediyl or oxadiazolediyl is, for example, 2,4-, 2,5- or 3,5-pyridinediyl, 4,6-pyrimidinediyl, 2,4- or 2,5-thiophenediyl, 2,4- or 2,5-furandiyl, 2,4- or 2,5-thiazolediyl, 2,4- or 2,5-oxazolediyl, 2,5-thiadiazolediyl or 2,5-oxadiazolediyl.

A suitable value for R¹ when it is (2-5C) alkanoyl is, for example, acetyl, propionyl or to butyryl.

A suitable value for R² or R³ when it is (1-4C) alkyl is, for example, methyl, ethyl, propyl or isopropyl.

The substituent R³ may be attached to any available carbon atom of the rings, which form the group Q², including attachment to the carbon atom, which carries the substituent R².

When R² and R³ are linked to form a methylene, vinylene, ethylene or trimethylene group, with the substituent R² located on one of the carbon atoms alpha to the oxygen atom of the rings which form the group Q², the substituent R³ is preferably located on the other alpha carbon atom.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl) amine.

Preferably Q¹ is 2-oxoindolinyl, 2-oxo-1,2-dihydroquinolinyl, 2-oxo-1,2,3,4-tetrahydroquinolinyl or 2-oxo-2,3,4,5-tetrahydro-1H-benzo[b]azepinyl which may optionally bear one, two or three substituents selected from fluoro, chloro, methyl, ethyl, allyl and 2-propynyl;

X is thio, sulphinyl or sulphonyl;

Ar is 1,3-phenylene which may optionally bear one or two fluoro substituents or Ar is 2,4- or 2,5-thiophenediyl or 2,4- or 2,5-thiazolediyl; and

Q² is a group of the formula (I) I wherein R¹ is hydrogen;

R² is methyl or ethyl; and

R³ is hydrogen or methyl;

or R² and R³ are linked to form an ethylene group.

Preferably the ether derivative of formula (I) is:

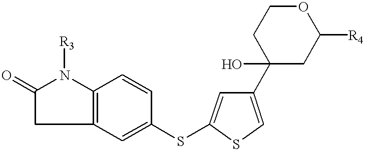

wherein R³ and R⁴ are independently (1-4C) alkyl.

Preferably R³ and R⁴ are both methyl.

Preferably the ether derivative of formula (I) is selected from:

(2S, 4R)-4-hydroxy-2-methyl-4-[2-(1-methyl-2-oxoindolin-5-ylthio)thien-4-yl]-tetrahydropyran;

(2S, 4S)-4-hydroxy-2-methyl-4-[2-(1-methyl-2-oxoindolin-5-ylthio)thien-4-yl]-tetrahydropyran;

(2R, 4R)-4-hydroxy-2-methyl-4-[2-(1-methyl-2-oxoindolin-5-ylthio)thien-4-yl]-tetrahydropyran;

(2R, 4S)-4-hydroxy-2-methyl-4-[2-(1-methyl-2-oxoindolin-5-ylthio)thien-4-yl]-tetrahydropyran.

Most preferably the ether derivative is (2S, 4R)-4-hydroxy-2-methyl4-[2-(1-methyl-2-oxoindolin-5-ylthio)thien4-yl]tetrahydropyran.

A compound of the invention comprising an ether derivative of the formula (I), or a pharmaceutically acceptable salt thereof, may be prepared as described in U.S. Pat. No. 5,512,594 which is incorporated by reference herein.

The invention further provides a pharmaceutical composition for treatment or prevention of COPD, which contains a compound of formula (I), or a metabolite thereof, or a pharmaceutically acceptable salt thereof.

The composition may be in a form suitable for oral use, for example a tablet, capsule, aqueous or oily solution, suspension or emulsion; for topical use, for example a cream, ointment, gel or aqueous or oily solution or suspension; for nasal use, for example a snuff, nasal spray or nasal drops; for vaginal or rectal use, for example a suppository; for administration by inhalation, for example as a finely divided powder such as a dry powder (for example using air or a halocarbon as carrier), a microcrystalline form or a liquid aerosol; for sublingual or buccal use, for example a tablet or capsule; or for parenteral use (including intravenous, subcutaneous, intramuscular, intravascular or infusion), for example a sterile aqueous or oily solution or suspension. In general the above compositions may be prepared in a conventional manner using conventional excipients.

The amount of active ingredient (that is an ether derivative of the compound of formula (I), or a pharmaceutically acceptable salt thereof) that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of one or more excipients, and the amount of active ingredient may vary from about 5 to about 98 percent by weight of the total composition.

In general, satisfactory results will be obtained when the pharmaceutical composition is administered such that the total daily dosage of the ether derivative of the compound of formula (I) is in the range from 1 mg to about 500 mg of an active ingredient. Preferably the total daily dosage is between 30 and 100 mg.

In a fiber aspect the invention provides the use of an ether derivative of the compound of formula (I), or a metabolite thereof, or a pharmaceutically acceptable salt thereof, for the treatment or prevention of COPD in a mammal (especially a human).

The invention also provides a method of treating or preventing COPD in a mammal (especially a human) which comprises administering (for example orally) to a patient a compound of formula (I), or a metabolite thereof, or a pharmaceutically acceptable salt thereof.

1. The use of an ether derivative of a compound of formula (I):
Q¹—X—Ar—Q² (I)
wherein Q¹ is a 9-, 10- or 11-membered bicyclic heterocyclic moiety containing one or two nitrogen heteroatoms and optionally containing a further heteroatom selected from nitrogen, oxygen and sulphur, and Q¹ may optionally bear up to four substituents selected from halogeno, hydroxy, cyano, formyl, oxo, thioxo, (1-4C) alkyl, (3-4C) alkenyl, (3-4C) alkynyl, (1-4C) alkoxy, fluoro-(1-4C) alkyl, hydroxy-(1-4C) alkyl, (2-5C) alkanoyl, phenyl, benzoyl and benzyl, and wherein said phenyl, benzoyl and benzyl substituents may optionally bear one or two substituents selected from halogeno, (1 -4C) alkyl and (1-4C) alkoxy;
X is oxy, thio, sulphinyl or sulphonyl;
Ar is phenylene, pyridinediyl, pyrimidinediyl, thiophenediyl, furandiyl, thiazolediyl, oxazolediyl, thiadiazolediyl or oxadiazolediyl which may optionally bear one or two substituents selected from halogeno, cyano, trifluoromethyl, hydroxy, amino, (1-4C) alkyl, (1-4C) alkoxy, (1-4C) alkylamino and di-(1-4C) alkylamino;
and Q² is selected from the groups of the formulae II and III: 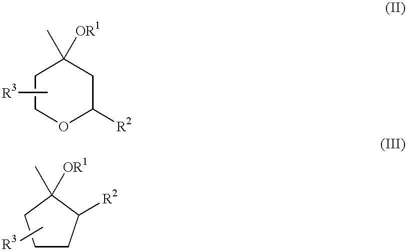
wherein
R¹ is hydrogen, (2-5C) alkanoyl or benzoyl, and wherein said benzoyl group may optionally bear one or two substituents selected from halogeno, (1-4C) alkyl and (1-4C) alkoxy;
R² is (1-4C) alkyl; and
R³ is hydrogen or (1-4C) alkyl;
or R² and R³ are linked to form a methylene, vinylene, ethylene or trimethylene group;
or a metabolite thereof, or a pharmaceutically acceptable salt thereof, for the manufacture of a medicament for use in the treatment or prevention of COPD in a mammal (especially a human).

2. Use according to claim 1 wherein Q¹ is 2-oxoindolinyl, 2-oxo-1,2-dihydroquinolinyl, 2-oxo-1,2,3,4-tetrahydroquinolinyl or 2-oxo-2,3,4,5-tetrahydro-1H-benzo[b]azepinyl which may optionally bear one, two or three substituents selected from fluoro, chloro, methyl, ethyl, allyl and 2-propynyl;
X is thio, sulphinyl or sulphonyl;
Ar is 1,3-phenylene which may optionally bear one or two fluoro substituents or Ar is 2,4- or 2,5-thiophenediyl or 2,4- or 2,5-thiazolediyl; and
Q² is a group of the formula (I) I wherein R¹ is hydrogen;
R² is methyl or ethyl; and
R³ is hydrogen or methyl;
or R² and R³ are linked to form an ethylene group.

3. Use according to claim 1 or 2 wherein the ether derivative of formula (I) is: 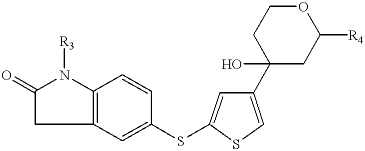
wherein R³ and R⁴ are independently (1-4C) alkyl.

4. Use according to any one of claims 1 to 3 wherein R³ and R⁴ are both methyl.

5. Use according to any one of claims 1 to 4 wherein the ether derivative of formula (I) is selected from:
(2S,4R)-4-hydroxy-2-methyl-4-[2-(1-methyl-2-oxoindolin-5-ylthio)thien-4-yl]-tetrahydropyran;
(2S,4S)-4-hydroxy-2-methyl-4-[2-(1-methyl-2-oxoindolin-5-ylthio)thien-4-yl]-tetrahydropyran;
(2R,4R)-4-hydroxy-2-methyl-4-[2-(1-methyl-2-oxoindolin-5-ylthio)thien-4-yl]-tetrahydropyran;
(2R,4S)-4-hydroxy-2-methyl-4-[2-(1-methyl-2-oxoindolin-5-ylthio)thien-4-yl]-tetrahydropyran.

6. Use according to any one of claims 1 to 5 wherein the ether derivative is (2S, 4R)-4-hydroxy-2-methyl-4-[2-(1-methyl-2-oxoindolin-5-ylthio)thien-4-yl]tetrahydropyran.

7. A pharmaceutical composition for treatment or prevention of COPD which contains a compound of formula (I) as defined in any one of the preceding claims, or a metabolite thereof, or a pharmaceutically acceptable salt thereof.

8. Use of an ether derivative of the compound of formula (I) as defined in any one of the preceding claims for the treatment or prevention of COPD in a mammal (especially a human).

9. A method of treating or preventing COPD in a mammal (especially a human) which comprises administering to a patient a compound of formula (I) as defined in any one of the preceding claims, or a metabolite thereof, or a pharmaceutically acceptable salt thereof.
